(19) United States
(12) Patent Application Publication
(10) Pub. No.: US 2012/0215063 A1
(43) Pub. Date: Aug. 23, 2012

(54) PELVIC FLOOR REPAIR SYSTEM

(75) Inventors: Henry Holsten, Covington, GA (US); Robert Young, Bogart, GA (US); Justin Wolfe, Lawrenceville, GA (US); Doug Evans, Snellville, GA (US)

(73) Assignee: C. R. BARD, INC, Murray Hill, NJ (US)

(21) Appl. No.: 13/391,214

(22) PCT Filed: Aug. 18, 2010
(86) PCT No.: PCT/US10/45943

§ 371 Date: May 7, 2012

Related U.S. Application Data
(60) Provisional application No. 61/234,811, filed on Aug. 18, 2009.

(51) Int. Cl. A61F 2/04

(52) U.S. Cl. 600/37

(57) ABSTRACT

Described herein are devices and methods comprising a system for tissue reinforcement, such as stabilization of fascial structures of the pelvic floor in vaginal wall prolapse. The surgical treatment can provide a mechanical support or bridging material for a fascial defect. An implant with four arms, for example two proximal arms and two distal arms, can provide post-surgical adjustability of the graft position, enabling tension free placement of the implant. In addition, a four-point fixation of the implant with apical support can ensure a suitable anatomical repair. Furthermore, instrumentation is disclosed herein, including an introducer device capable of fixing a wide variety of materials, such as mesh arms and suture, to tissue.

This application is related to and claims benefit of priority to co-pending U.S. Provisional Patent Application No. 61/234,811, filed Aug. 18, 2009, and titled “PELVIC FLOOR REPAIR SYSTEM,” the contents of which are incorporated herein by reference in their entirety.

According to certain embodiments, the present disclosure relates to systems and methods for pelvic floor repair. Pelvic organ prolapse disorders typically result from weakness in or damage to normal pelvic organ support structures. Pelvic organ prolapse disorders can include cystoceles (whether ventral or lateral), rectoceles, enteroceles, urethreoceles, hysteroceles, vaginal vault prolapse, uterine prolapse, and/or any other prolapse or combination of such disorders. Also, the system may be configured to treat pelvic floor disorders of both males and females. These disorders can result from childbirth, removal of the uterus, connective tissue defects, postmenopausal atrophy, and heavy labor.

The prolapse repair system in accordance with the present disclosure can be used for tissue reinforcement and long-lasting stabilization of fascial structures of the pelvic floor, wherein surgical treatment is intended either as mechanical support or bridging material for the fascial defect. This includes, but is not limited to the following procedures: vaginal wall prolapsed repairs including anterior and posterior wall repairs, vaginal suspension, reconstruction of the pelvic floor and tissue repair.

According to various aspects of the disclosure, there is provided a prolapse repair system for tissue reinforcement and long-lasting stabilization of fascial structures of the pelvic floor in vaginal wall prolapse, where surgical treatment is intended either as mechanical support or bridging material for the fascial defect. An implant with four arms, for example two proximal arms and two distal arms, can provide post-surgical adjustability of the graft position, enabling tension free placement of the implant. In addition, a four-point fixation of the implant with apical support can ensure a suitable anatomical repair.

According to one aspect of the invention, there is provided a pelvic floor repair system comprising an introducer having a proximal end and a distal end, wherein a needle housing is located at the proximal end, a handle is located at the distal end, and a shaft connects the needle housing to the handle; the needle housing comprises a curved needle carrier having a proximal end and a distal end, wherein the curved needle pivots about the center of its axis of rotation, the needle housing further comprising a first aperture from which the distal end of the needle carrier exits upon actuation, and a second aperture; the handle comprises a manual actuation mechanism that drives movement of the distal end of the needle carrier to exit from the first aperture towards second aperture; an implant comprising a support portion and at least one extension connected to the support portion, wherein the at least one extension has a proximal end and a distal end, and the proximal end is connected to the support portion and the distal end is connected to a needle.

The at least one extension can comprise a tubular mesh arm defining a lumen extending at least partially therethrough. According to certain aspects of the disclosure, the needle is a bullet needle having a distal end configured to pierce tissue, a shoulder, and a proximal end that receives the distal end of the at least one extension. The proximal end of the bullet needle can be crimped around the distal end of the at least one extension.

The second aperture can include a needle catch configured to releasably capture the needle. Furthermore, the needle housing can further a manual release mechanism to release the needle from the needle catch.

According to various aspects of the disclosure, the introducer handle has a proximal end, a distal end, and a gripping region between the proximal and distal ends; the proximal end of the handle is connected to the distal end of the shaft; the shaft and the proximal end of the handle share the same longitudinal axis; and the longitudinal axis of the shaft and the longitudinal axis of the gripping region intersect at an angle ranging from 200° to 80°. The angle can be 170°, 160°, 150°, 140°, or any other suitable, ergonomic angle. The system can further comprise at least one introducer needle having a proximal end and a distal end and defining an inner lumen, the introducer needle further having an opening that provides access to the inner lumen; and a drive wire terminating in an implant coupling element, the drive wire being configured to be positioned within the inner lumen of the introducer needle, wherein the implant coupling element is extendable from the introducer needle opening to an extended position in which the implant coupling element is positioned outside of the inner lumen and retractable to a retracted position in which the implant coupling element is positioned within the inner lumen. According to one embodiment, the implant coupling element is a loop that can receive at least the distal end of the at least one extension.

According to one embodiment, the implant comprises two proximal extensions and two distal extensions, wherein at least two of the four extensions are each comprised of a tubular mesh defining a lumen extending at least partially therethrough. Furthermore, the implant can comprise two proximal extensions, each comprised of a tubular mesh defining a lumen extending at least partially therethrough; and two distal extensions, each comprised of a flat mesh.

According to one embodiment, the needle housing comprises a tissue engagement portion. The tissue engagement portion can have a concave shape that facilitates in-flow of tissue. According to one embodiment, the concave shape is defined by an arc. A line tangent to each end of the arc defines a first axis, and the shaft of the introducer defines a second axis. The first axis can intersect the second axis at an angle ranging from 15° to 80°, for example 20° to 70°, 30° to 60°, or 40°to 50°. Any other ergonomically effective angle can also be used.

According to one embodiment, the distal end of the needle carrier receives the proximal end of the needle. According to another embodiment, the shaft of the introducer comprises at least one bracket configured to receive and releasably secure the at least one implant extensions.

The present disclosure, according to certain non-limiting embodiments, relates to a pelvic floor repair system comprising an introducer having a proximal end and a distal end, wherein a needle housing is located at the proximal end, a handle is located at the distal end, and a shaft connects the needle housing to the handle; the needle housing comprises a curved needle carrier having a proximal end and a distal end, wherein the curved needle pivots about the center of its axis of rotation, the needle housing further comprising a first aperture from which the distal end of the needle carrier exits upon actuation, and a second aperture; the handle comprises a manual actuation mechanism that drives movement of the distal end of the needle carrier to exit from the first aperture towards second aperture; an implant comprising a support portion and at least one extension connected to the support portion, wherein the at least one extension has a proximal end and a distal end, and the proximal end is connected to the support portion and the distal end is connected to a needle; and a locking feature associated with at least one of the extensions, wherein the locking feature is configured to substantially lock the at least one extension in position relative to tissue.

The needle housing can comprise at least one external feature that provides tactile feedback to the user. The locking feature can be a securement ring, through which the at least one extension is received. The internal diameter of the securement ring can comprises at least one feature that substantially permits movement of the extension through the ring in one direction, and substantially inhibits movement of the extension through the ring in the opposite direction.

### BRIEF DESCRIPTION OF THE DRAWINGS

The disclosed systems and methods can be better understood with reference to the following drawings. The components in the drawings are not necessarily to scale.

FIG. 1 is a perspective view of one embodiment of an introducer.

FIG. 2A is a cutaway view of a needle housing with the needle carrier in the retracted configuration.

FIG. 2B is a cutaway view of a needle housing with the needle carrier in the extended configuration.

FIG. 2C is a cutaway view of a needle housing with the bullet needle seated in the needle carrier.

FIG. 2D is a cutaway view of a needle housing with the bullet needle in the needle catch.

FIG. 2E is a cutaway view of a needle housing, with the needle carrier in the retracted position and the bullet needle in the needle catch.

FIGS. 2F-2G is a close up of the positioning of the bullet needle in the needle carrier.

FIG. 2H is a close up of the bullet needle being released from the needle catch.

FIG. 2I is a cutaway view of the needle housing.

FIG. 3 is a cutaway view of the introducer handle.

FIG. 4 is a plan view of an implant suitable for an anterior pelvic floor repair procedure.

FIG. 5 is a plan view of an implant suitable for a posterior pelvic floor repair procedure.

FIG. 6 is a close up view of a bullet needle.

FIG. 7 is a close up view of a securement ring.

FIG. 8 is a close up view of a support extension encircled by a securement ring.

FIG. 9A is a perspective view of a deployment tool.

FIG. 9B is a cutaway view of a deployment tool.

FIG. 9C is a cutaway view of the distal end of the deployment tool.

FIG. 10 is a cutaway view of a deployment tool.

FIG. 11A is a perspective view of a snare introducer needle, with the snare partially deployed.

FIG. 11B is a perspective view of a snare introducer needle with the snare in the fully retracted position.

FIG. 12 is a perspective view of the needle housing seated against the sacrospinous ligament.

FIG. 13 is a perspective view of an implanted anterior support member.

FIG. 14A is a close up view of a deployment tool pressed against the sacrospinous ligament.

FIG. 14B is a close up view of a suspension member once it has been drawn through the sacrospinous ligament.

### DESCRIPTION

The following description and accompanying figures, which describe and show certain embodiments, are made to demonstrate several possible configurations that a pelvic floor repair system can take including to include various aspects and features of the present disclosure. In some of the figures, an illustrated embodiment is shown in use in a pelvic cavity of a patient. The illustration of the medical device in this context is not intended to limit the disclosed aspects and features of the invention to the specified embodiments or usage only within the pelvic cavity. Those of skill in the art will recognize that the disclosed aspects and features of the invention are not limited to any particular embodiment of a surgical system or device.

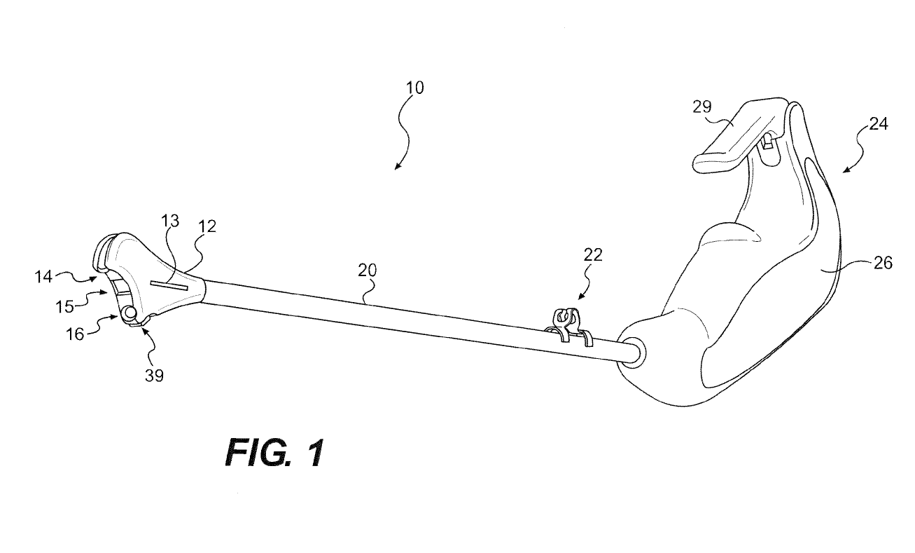

According to various embodiments, the introducer 10 illustrated in FIG. 1 is suitable for implanting a medical device, such as suture or an implant, for example a pelvic floor implant. The introducer includes a handle 24 having a gripping area 26 and a trigger 29. According to one embodiment, the trigger 29 can be compressed with the user's fingers while the palm of the user's hand is wrapped around the gripping area 26. The user's thumb is typically resting at the distal end of handle 24. The distal end of the handle is connected to shaft 20, and the distal end of shaft 20 is connected to needle housing 12. The needle housing 12 can include at least one tactile feedback feature 13, which allows the user to tactilely locate the position and orientation of the needle housing 12 when the distal end of the introducer is not visible (e.g., situated within a patient's body during a surgical procedure).

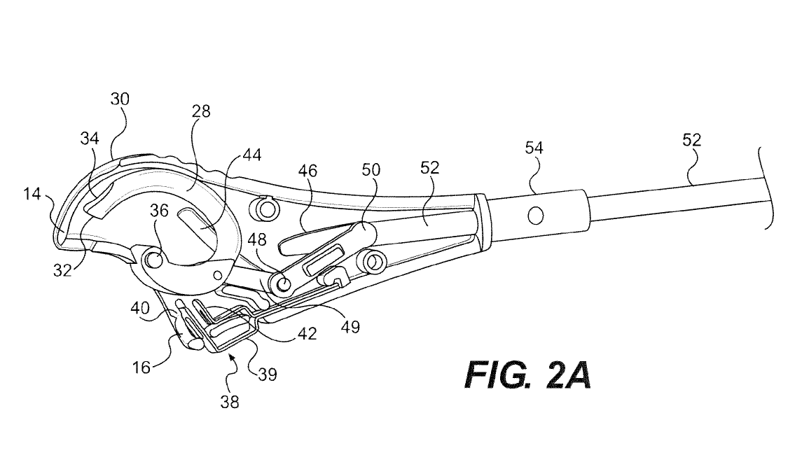

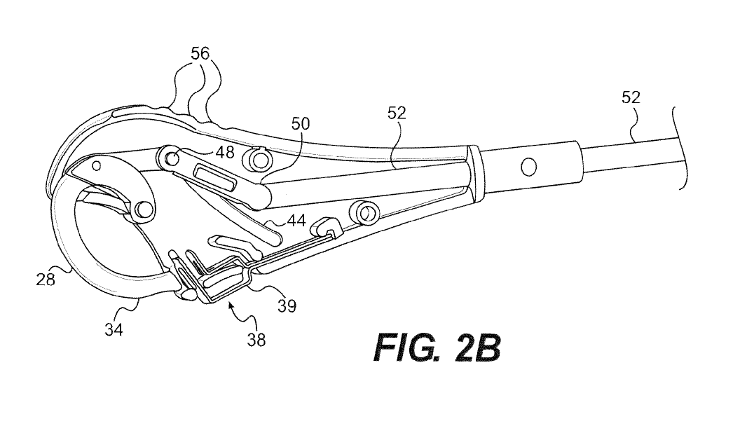

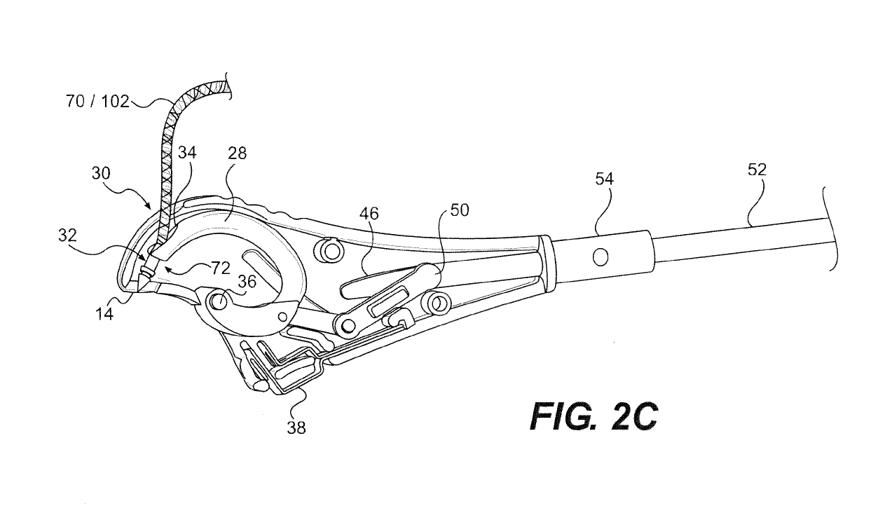

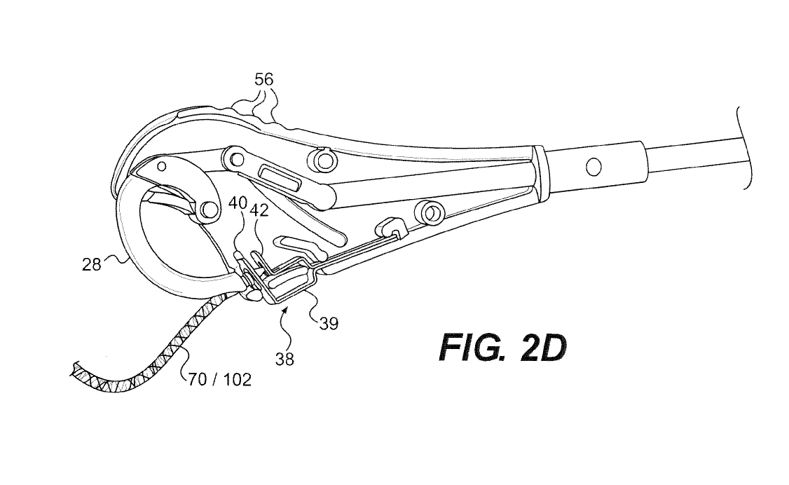

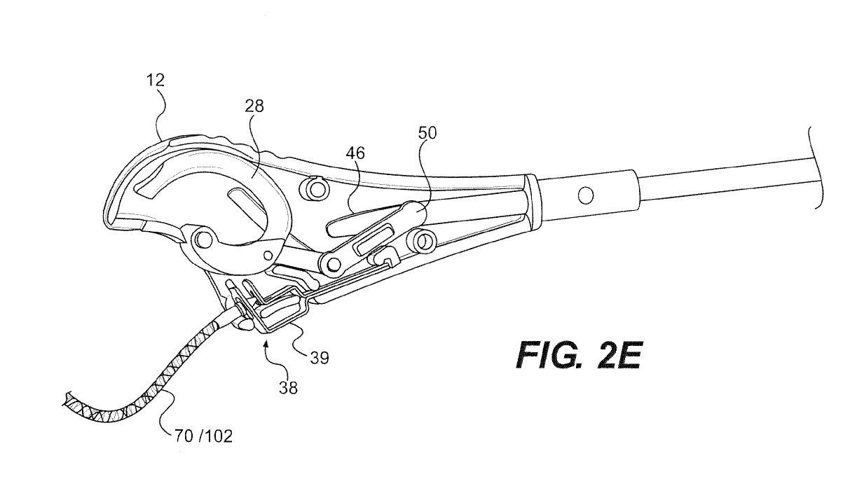

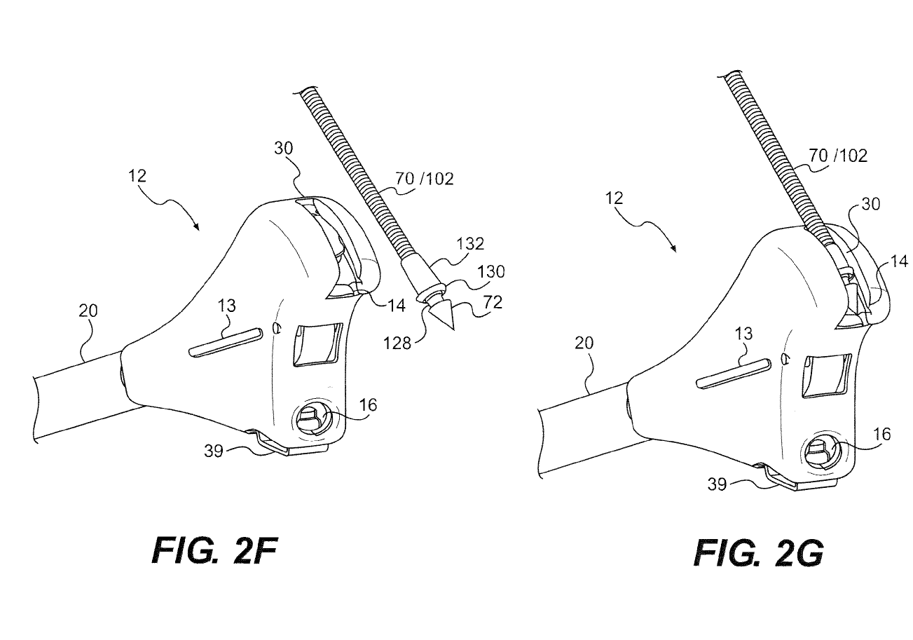

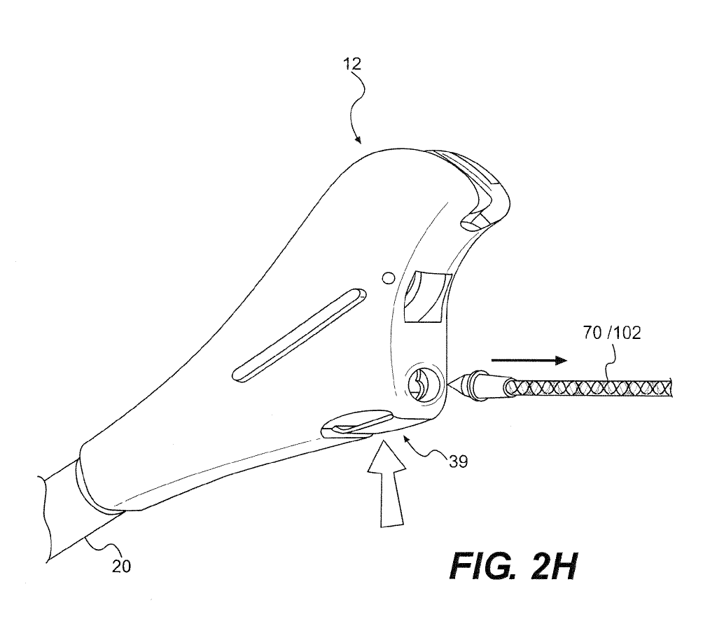

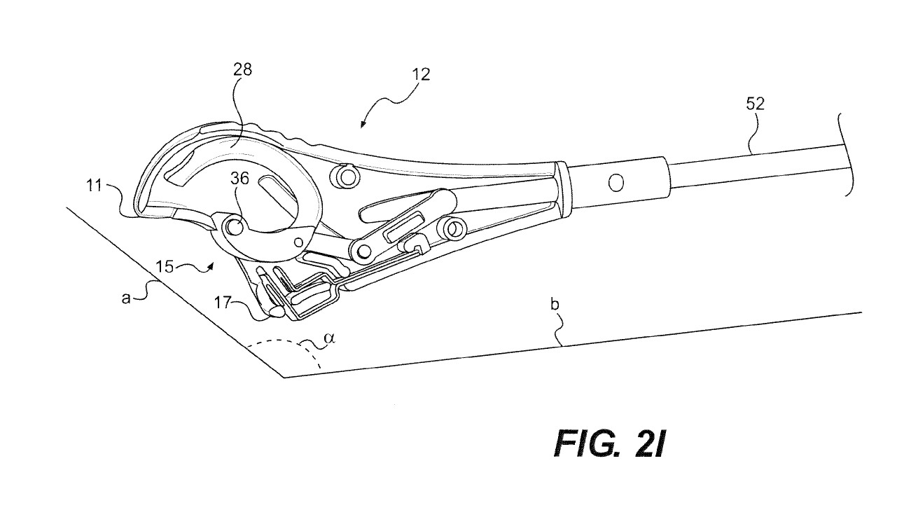

According to one embodiment, needle housing 12 also includes a shaped tissue engagement portion 15 configured to allow tissue such as, for example, ligament, tendon, muscle, or any other tissue to “tent up” or “pillow in” to the shape. By way of example, the shaped tissue portion can have a concave shape. The needle housing 12 also includes a first aperture 14, through which the distal end of a needle can exit, and a second aperture 16 that can receive the distal end of a needle. A needle catch (38 in FIG. 2B), which is an active, as opposed to passive, catch can release the needle when tab 39 is depressed.

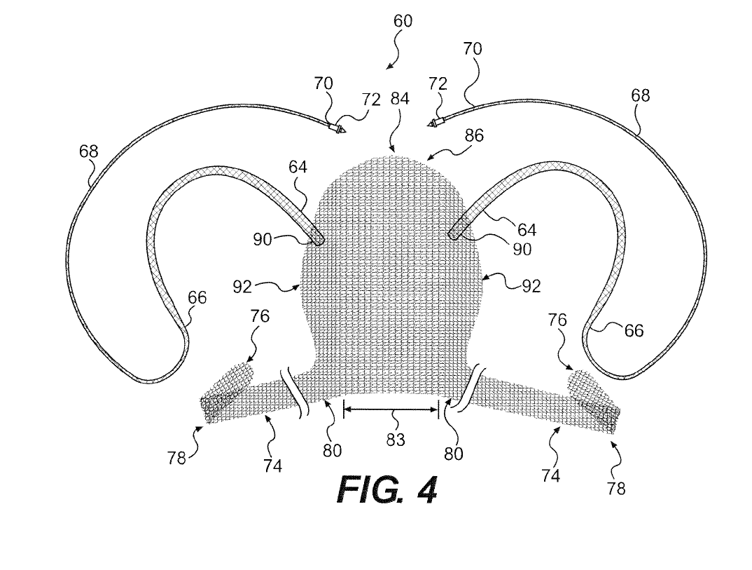

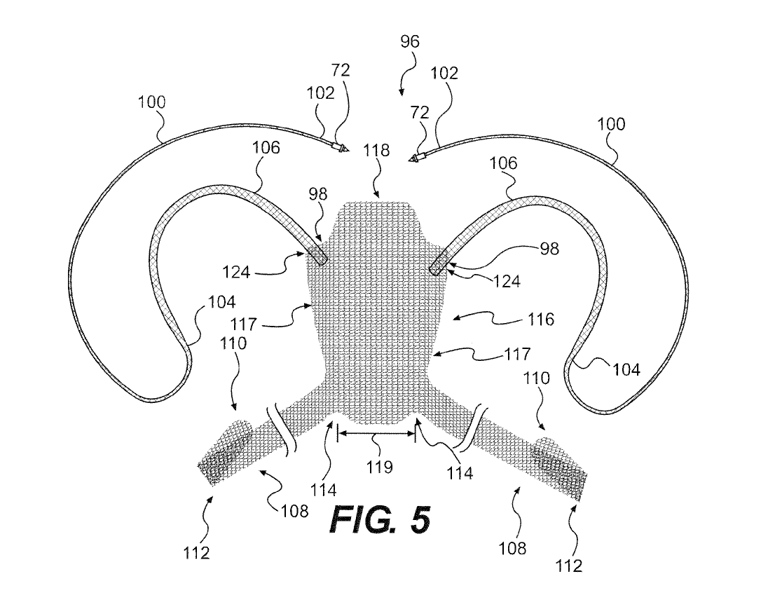

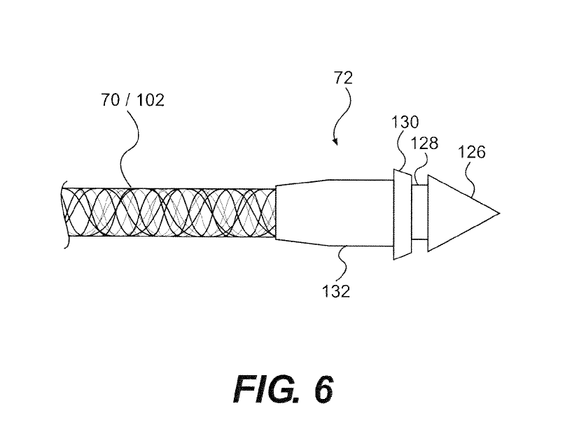

A cutaway view of needle housing 12 is illustrated in FIG. 2A. Drive shaft 52 extends through the proximal end 54 of the housing. The distal end of drive shaft 52 is connected to the drive linkage 50 via a pin (not shown) that is perpendicular to the longitudinal axis of the drive shaft 52. The pin extends into, and moves axially along, recessed cam surface 46. A second pin 48 connects drive linkage 50 to center linkage 49, and the second pin 48 moves along recessed cam surface 44. Such features define a splined (composite) curve that translates the linear motion of the drive shaft 52 to rotational motion of the needle carrier 28. The center linkage 49 can be directly or indirectly connected to needle carrier 28, which pivots about axis post 36. The proximal end of a driving element, for example a needle, such as a surgical needle or a bullet needle 72 (FIG. 6) can be seated within distal end 32 of needle carrier 28. Aperture 34 is configured to receive a filamentary element, such as a suture, strip of mesh, or tubular mesh arm 68 in FIG. 4 or 100 in FIG. 5 connected to the proximal end of a driving element, such as a needle. Slot 30 receives the filamentary element trailing from aperture 34.

Active needle catch 38 receives at least a portion of a needle, for example the distal end of a needle, when needle carrier 28 completes its rotation around axis post 36. Referring to bullet needle 72 in FIG. 6 as an example, the distal end of the needle 72 is first received within aperture 16, and further movement of the conical head 126 moves collar 40 upwards towards axis post 36. As illustrated in FIG. 2D, once conical head 126 clears collar 40, collar 40 snaps back down and is received within groove 128 of needle 72. The tip of the needle is then received within second collar 42. The shoulder characterized by the step between conical head 126 and groove 128 (FIG. 6) prevents unintentional dislodgment of bullet needle 72 from collar 40. As shown in FIG. 2E, once the needle 72 is captured within catch 38, trigger 29 is released and needle carrier 28 retracts into needle housing 12. To release the needle from catch 38, collar 40 is released from groove 128 towards axis post 36 by pressing tab 39 (FIG. 2H).

FIG. 2B shows needle carrier 28 in its fully extended position, having been extended by depressing trigger 29 (FIG. 1). Drive shaft 52 is urged distally, resulting in movement of the drive and center linkages 50 and 49, respectively, and then rotation of needle carrier 28 about axis post 36. Also shown in FIG. 2B is tactile feature 56, allowing a user to manually locate and identify the orientation of the needle housing 12 when it is out of the field of view.

Although certain embodiments relate to the use of introducer 10 for throwing mesh arms through ligaments, the present disclosure is not so limited. The introducer 10 can also suitably be used as a suturing instrument, and thus used in a wide variety of surgical instruments. In one embodiment, the introducer 10 has a spool (not shown) or other suitable feature configured to hold a length of suture. According to certain embodiments, the introducer can be employed to implant slings, including urethral slings, in a patient. A discussion of urethral slings and methods for their implantation can be found, for example, in commonly owned U.S. patent application Ser. No. 12/441,123, filed Sep. 12, 2007, titled URETHRAL SUPPORT SYSTEM; U.S. patent application Ser. No. 12/269,749, filed Nov. 12, 2008, titled ADJUSTABLE TISSUE SUPPORT MEMBER; and U.S. patent application Ser. No. 12/093,493, filed Nov. 14, 2006, titled SLING ANCHOR SYSTEM, the disclosures of which are each incorporated herein by reference in their entirety.

In FIG. 2C, bullet needle 72 is situated within distal end 32 of needle carrier 28. Distal end 70/102 of extension 68/100 is received through aperture 34 and slot 30. FIGS. 2F-2G illustrate loading of bullet needle 72 into needle housing 12 by placing needle 72 and distal end 70/102 of extension 68/100 into slot 30 of the housing 12. According to various embodiments, tactile and/or audible feedback (such as a click) may be received by the user once the needle 72 is properly seated within housing 12 (FIG. 2G).

As illustrated in FIG. 2I, tissue engagement portion 15 can have a certain orientation with respect to the longitudinal axis defined by drive shaft 52. Line a in FIG. 2I is tangent to points 11 and 17 on the needle housing 12, and the lines a and b form an angle a. According to various embodiments, the angle can range from 100° to 170°. For example, the angle can range from 120° to 160°, including 135°. Any angle suitable for engaging and receiving tissue can be used.

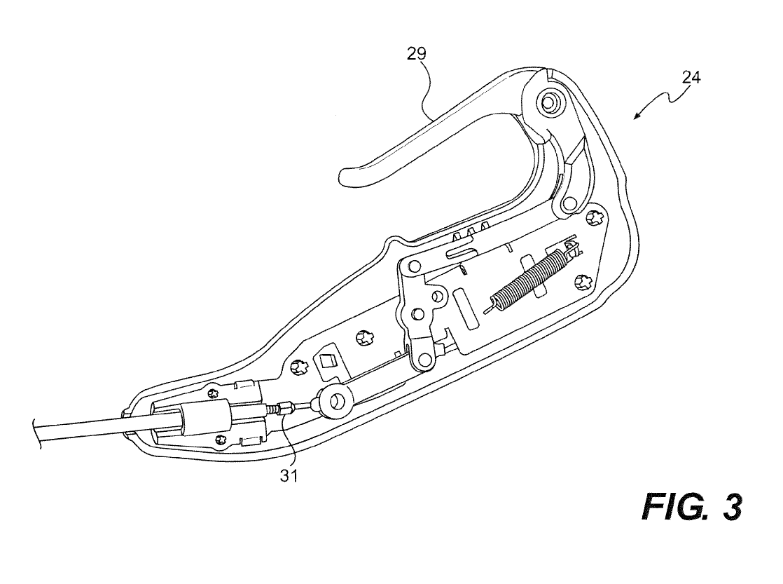

FIG. 3 is a cutaway view of handle 24, illustrating one possible embodiment of a mechanism that translates actuation of trigger 29 to movement of drive shaft 52. Lead screw 31 can be rotated to adjust the length of throw of the needle carrier 28. For example, rotation of the lead screw 31 in a clockwise direction will shorten the throw, and rotation in a counterclockwise direction with lengthen the throw of the needle carrier 28. Lead screw 31 is but one embodiment of an adjusting feature; other known suitable adjusting features performing substantially the same function can also be used.

FIG. 4 illustrates one embodiment of an implant 60 suitable for an anterior prolapse repair. Implant 60 comprises a support member 86, two proximal extensions (or arms) 68, and two distal extensions (or arms) 74. Throughout this disclosure, the term “extension,” “arm,” and “filamentary element” can be used interchangeably. Support member 86 is configured to support tissue or an organ such as, for example, a bladder. According to various embodiments, the tissue support member 86 can comprise a central portion 83 and two lateral portions 92. In some embodiments, the support member 86 comprises a woven mesh. The central portion 83 can have a mesh construction that is different from the lateral portions 92. For example, the central portion 83 can be constructed from a mesh that has a density lower than the density of the two lateral portions 92. According to various embodiments, the longitudinal axis of the support portion can be defined by a midline indicator 84.

Implant 60 can also comprise at least one arm that holds the support portion 86 in a desired position. As illustrated in FIG. 4, implant 60 can comprise two proximal arms 68. The proximal arm 68 has a distal end 70 terminating in a bullet needle 72, and a proximal end 64 connected at joint 90 to support member 86. The distal end 70 of proximal arm 68 can be connected to bullet needle 72 by any suitable method known in the art, including swaging, crimping, gluing, etc. Proximal end 64 can be connected to support member 86 by any suitable method, including but not limited to sewing, gluing, and ultrasonic welding. Distal arm 68 can have a varying diameter along its length. For example, proximal end 64 can have a first diameter that necks down at 66 to a second diameter. The diameter change can be stepped, or gradual. According to certain embodiments, distal end 70 has a first diameter, and proximal end 64 has a second diameter greater than the first diameter, facilitating dilation of the path of arm 68 through tissue.

According to certain embodiments, proximal arm 68 can be a suture, a strip of mesh, or any other material, including biomaterials, that can be drawn through tissue. According to one embodiment, proximal arm 68 is constructed of a tubular mesh defined by a lumen extending at least partially therethrough. A tubular mesh construct can be advantageous by permitting tissue in-growth into its pores upon implantation. In addition, a tubular mesh arm can slide through a ligament, such as a sacrospinous ligament, like a suture. Furthermore, the tubular mesh arm can act as a dilator. The tubular mesh can be constructed by methods known in the art of mesh knitting.

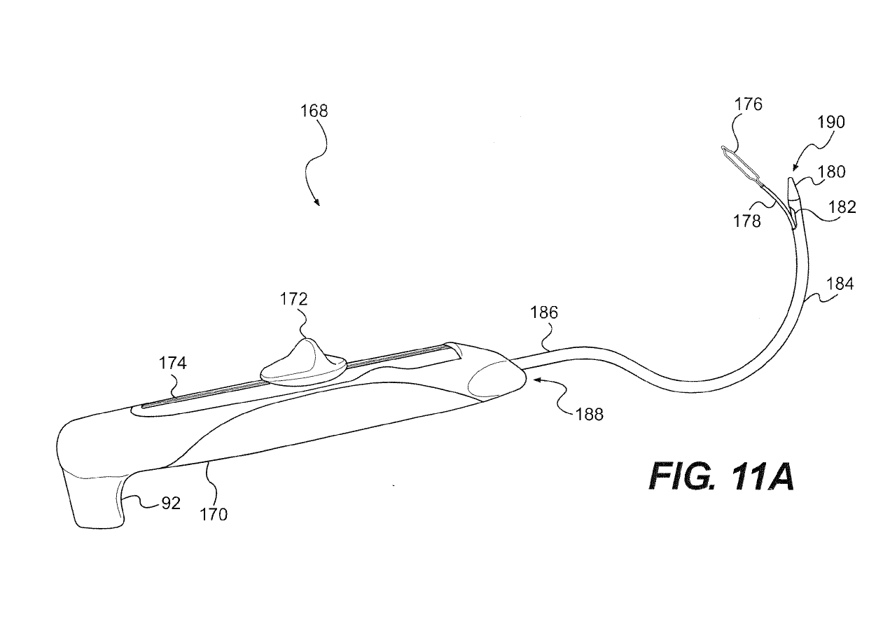

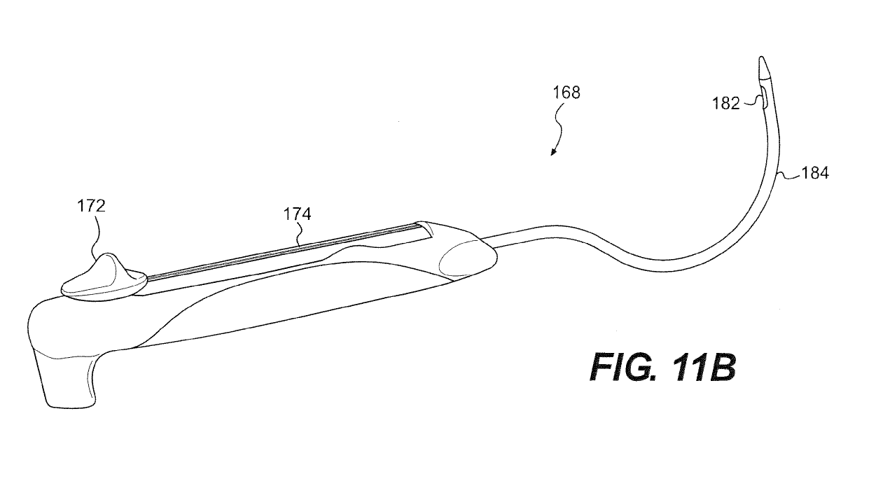

As illustrated in FIG. 4, implant 60 can also comprise two distal arms 74, having a proximal end 80 and a distal end 76. According to various embodiments, the proximal arms can be folded at a location 78, which is proximate to the distal end 76. The folded feature can assist the user in determining whether the implant 60 is corrected oriented in a patient, and whether the arm 74 is twisted within the patient. In addition, the fold 78 can facilitate proper capture of the arm 74 by a snare introducer 168 (FIGS. 11A-11B). Arms 74 are illustrated as flat mesh. However, the arms can also be sutures, tubular mesh, cord, or any other material that can be drawn through tissue.

FIG. 5 illustrates one embodiment of an implant 96 suitable for a posterior prolapse repair. Implant 96 comprises a support member 116, two proximal extensions (or arms) 100, and two distal extensions (or arms) 108. Support member 116 is configured to support tissue or an organ such as, for example, the rectum. According to various embodiments, the tissue support member 116 can comprise a central portion 119 and two lateral portions 117. In some embodiments, the support member 116 comprises a woven mesh. The central portion 119 can have a mesh construction that is different from the lateral portions 117. For example, the central portion 119 can be constructed from a mesh that has a density lower than the density of the two lateral portions 117. According to various embodiments, the longitudinal axis of the support portion can be defined by a midline indicator 118.

Implant 96 can also comprise at least one arm that holds the support portion 116 in a desired position. As illustrated in FIG. 5, implant 96 can comprise two proximal arms 100. The proximal arm 100 has a distal end 102 terminating in a bullet needle 72, and a proximal end 98 connected at joint 124 to support member 116. The distal end 102 can be connected to bullet needle 72 by any suitable method known in the art, including swaging, crimping, gluing, etc. Proximal end 98 can be connected to support member 116 by any suitable method, including but not limited to sewing, gluing, and ultrasonic welding. Proximal arm 100 can have a varying diameter along its length. For example, proximal end 98 can have a first diameter that necks down at 104 to a second diameter. The diameter change can be stepped, or gradual. According to certain embodiments, distal end 102 has a first diameter, and proximal end 98 has a second diameter greater than the first diameter, facilitating dilation of the path of arm 100 through tissue.

According to certain embodiments, arm 100 can be a suture, a strip of mesh, or any other material that can be drawn through tissue. According to one embodiment, arm 100 is constructed of a tubular mesh defined by a lumen extending at least partially therethrough. A tubular mesh construct can be advantageous by permitting tissue ingrowth into its pores upon implantation. The tubular mesh can be constructed by methods known in the art of mesh knitting.

As illustrated in FIG. 5, implant 96 can also comprise two distal arms 108, having a proximal end 114 and a distal end 110. According to various embodiments, the proximal arms can be folded at a location 112, which is proximal the distal end 110. The folded feature can assist the user in determining whether the implant 96 is correctly oriented in a patient, and whether the arm 108 is twisted within the patient. In addition, the fold 112 can facilitate proper capture of the distal end 110 of distal arm 108 by a snare introducer 168 (FIGS. 11A-11B). Arms 108 are illustrated as flat mesh. However, the arms can also be sutures, tubular mesh, cord, biomaterials, wire, or any other material that can be drawn through tissue.

The midline indicators 84 and 118 can be formed in any suitable manner, as the present disclosure is not limited in this respect. In one embodiment, the midline indicator is added after the implant is formed using a suitable ink or paint. In another embodiment, the color band results from a differently colored strand of knitted mesh that is knitted in during knitting of the implant. Alternatively, the midline indicator may be formed as a separate element that is subsequently woven through the interstices of a mesh implant. In one embodiment, the midline indicator is blue; however, any other suitable color(s) may be employed, as the present disclosure is not limited in this respect. Any indicia identifying the midline of the support portion can be used.

It should be appreciated that the present disclosure is not limited to repair of prolapsed organs. Rather, the system may be used to aid in the repair of other tissue or muscle defects or incisions, including but not limited to hernias or torn ligaments, or may be used as a graft, an anastomosis wrap or other gastric band, all whether located in the pelvic area, abdominal area, or other areas, as the present disclosure is not limited in this respect.

According to various embodiments, the implants 60 and 96 comprise a flexible material. The implant material can be formed of a synthetic material, a natural material, or a combination of both synthetic and natural materials. Irrespective of the material used to construct the implants, the implants are highly flexible yet have the strength needed for tension-free fixation.

According to certain embodiments, the support members 86 and 116 are formed from a sheet of synthetic material that may be formed from a sheet of knitted polypropylene monofilament mesh fabric, such as Bard Mesh available from C. R. Bard, Inc. of Murry Hill, N.J. When implanted, the polypropylene mesh promotes tissue or muscle ingrowth into and around the mesh structure. According to another aspect of the disclosure, a hybrid implant formed of both synthetic and more natural materials is provided. In this manner, the synthetic material aids in providing the permanence of an implant as the underlying support, while the more natural material acts as buffer between the synthetic material and host tissue of the patient.

According to various embodiments, the natural material can be any suitable material, including but not limited to biologically-derived materials, such as cadaveric (human) or xenographic tissue (particularly of bovine or porcine origin)—for example dermis processed to make an acellular collagen scaffold or intestinal submucosa or other biological material and/or bioengineered materials. Collagen materials can be obtained from various sources such as that available from Cook Biomedical, Inc. under the name COOK SURGISIS. In one embodiment, the natural material comprises a cross-linked porcine dermal collagen material such as PELVICOL® surgical implant from Tissue Science Laboratories plc, or PELVISOFT® acellular biomesh produced by Sofradim. Other suitable bioengineered materials may be employed as the present disclosure is not limited in this respect.

According to certain embodiments, the support members 86 and 116 are formed from a non-absorbable monofilament polypropylene mesh having a knit pattern that allows the mesh to be cut in any shape without unraveling, and provides multidirectional elasticity and stretch. Implants suitable for tissue repair and that can be used in accordance with the present disclosure are discussed in U.S. patent application Ser. No. 12/282,641, filed Mar. 15, 2007, the disclosure of which is incorporated herein by reference in its entirety.

In accordance with the present disclosure, various types of needles can be used to insert the implant arms through tissue, including various types of surgical needles and bullet needles. FIG. 6 illustrates one example of a bullet needle in accordance with the present disclosure. The distal end 70/102 of the implant arm 68/100 terminates in the shaft 132 of bullet needle 72, and is held there by, for example, crimping the shaft around the mesh arm. Tip 126 is configured to have a sharp point to penetrate tissue. Groove 128 and shoulder 130 facilitate capturing the bullet needle 72 in a needle catch, such as needle catch 38 in FIG. 2A. The needles in accordance with the present disclosure can be made of any biocompatible material, including stainless steel, polypropylene, polytetrafluoroethylene, and bioabsorbable materials. Suitable compatible materials are well-known in the medical device field.

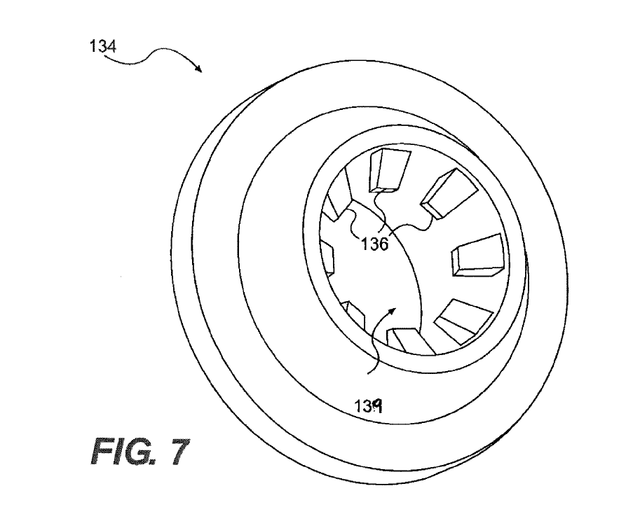

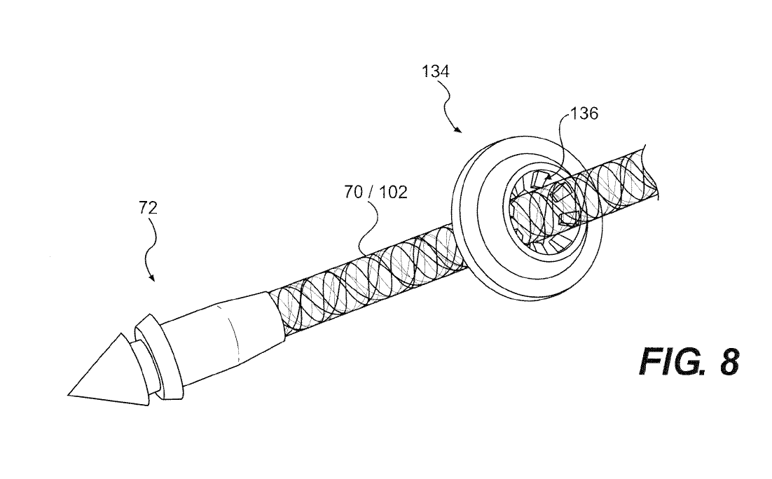

FIG. 7 illustrates a securement ring 134 configured to secure an implant arm in a substantially fixed location relative to tissue. The implant arm is received in inner diameter 139 as shown in FIG. 8, and flanges 136 permit movement of the arm through the washer in one direction, but resist movement in the opposite direction. The securement ring provides an additional measure of securement over the frictional forces of the tissue until tissue in-growth occurs.

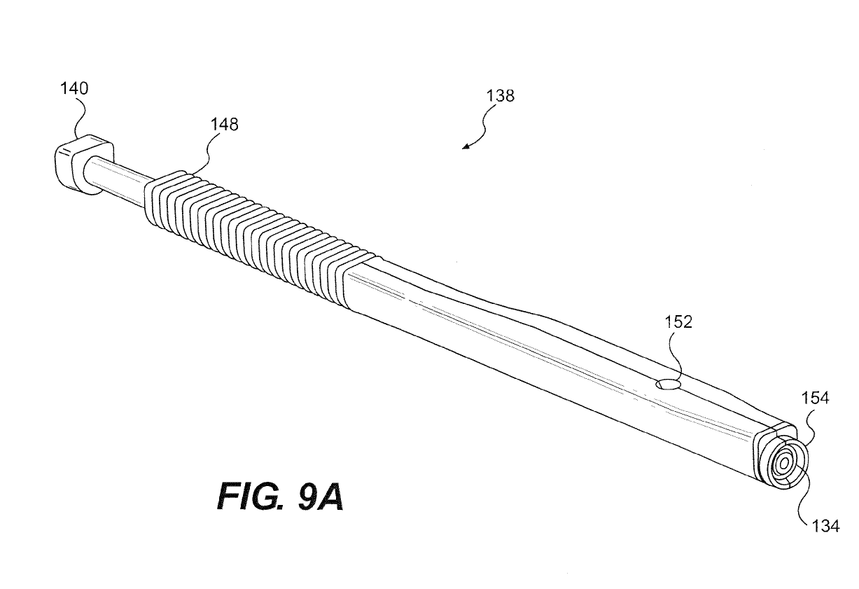

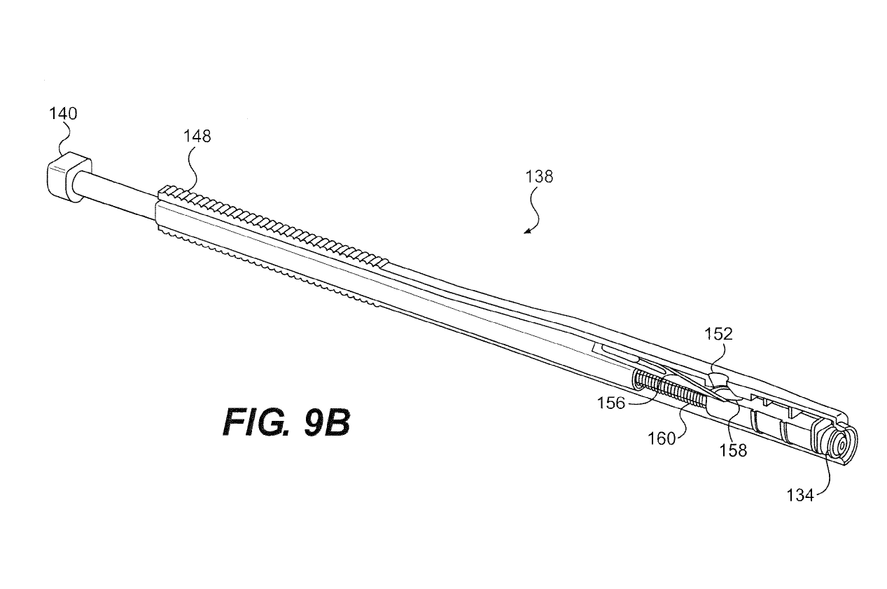

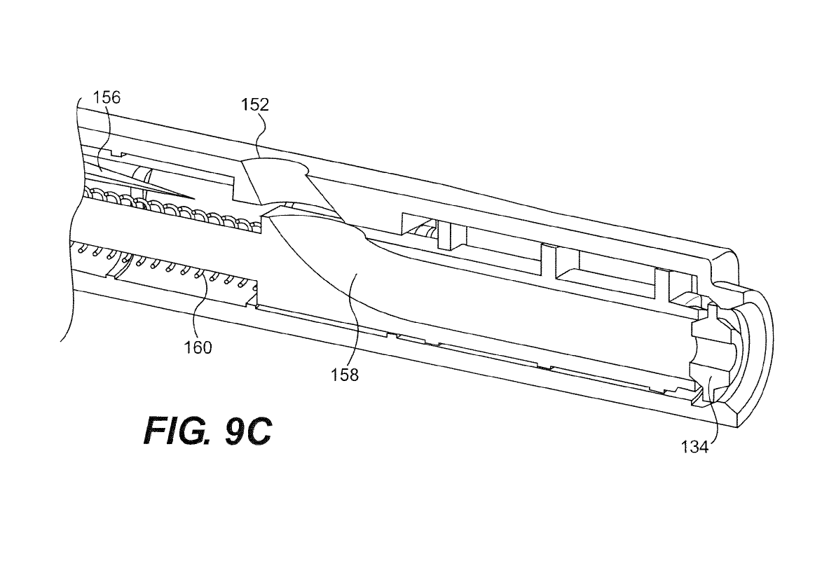

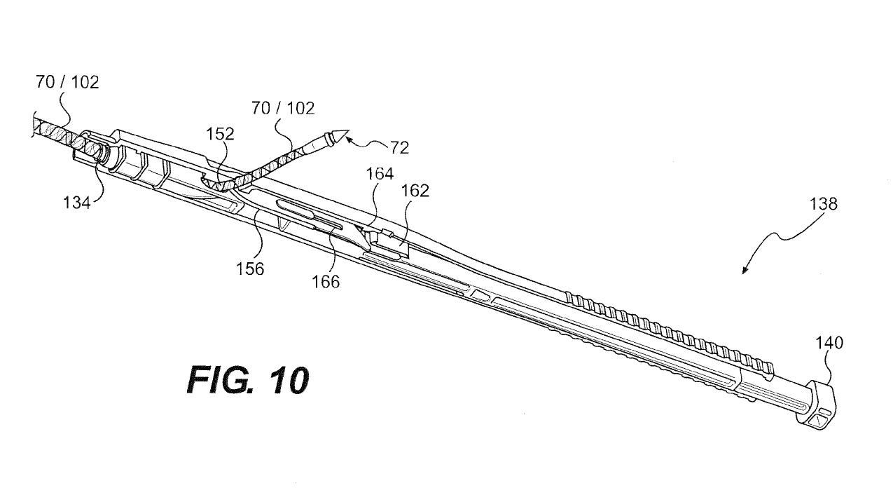

A deployment tool 138 preloaded with the securement ring 134 is illustrated in FIG. 9A. Once an implant arm has been positioned in tissue, for example the sacrospinous ligament, the distal end of the arm is fed through the center of securement ring 134, and out aperture 152. A cutaway view of the device 138 is shown in FIG. 9B. A user grips the device at the ribbed feature 148, push-button 140 is urged distally and against the bias of spring 160, and blade 156 severs the implant arm that extends through lumen 158. Simultaneously, securement ring 134 is deployed. A close-up of a cutaway view of the distal end of the deployment tool 138 is illustrated in 9C. FIG. 10 illustrates mesh arm 70/102 after it has been fed through securement ring 134 and lumen 158, exiting from aperture 152. Also shown in FIG. 10 is a lock-out feature having a catch 162 that irreversibly locks against abutment 164 once the deployment tool has been used.

FIGS. 11A and 11B illustrate a snare introducer 168 that can be used to deploy an implant. The introducer 168 includes a handle 170 and a needle having a shaft 184 with a proximal end 188 and a distal end 190. Proximal end 188 is joined to handle 170. Distal end 190 includes a tissue penetrating tip 180, and an aperture communicating with the lumen (not shown) defined by shaft 184. Drive wire 178 is fixed to button 172, so that as button 172 is moved in a proximal or distal direction along slot 174, wire 178 is also moved in a proximal or distal direction. An implant coupling element, such as loop 176, is configured to capture a portion of an implant and secure it as it is being manipulated. For example, an implant arm can be fed into loop 176, and the loop can then be withdrawn into aperture 182 by moving button 172 distally, thereby pulling at least a portion of the implant arm into the aperture.

FIG. 11B shows button 172 positioned in its distal-most position, such that loop 176 is disposed completely within shaft 184. Devices such as that of FIGS. 11A and 11B are described in U.S. patent application Ser. No. 12/282,641, filed Dec. 4, 2008; U.S. patent application Ser. No. 11/993,003, filed Jan. 22, 2008; U.S. patent application Ser. No. 11/993,089, filed Jun. 9, 2010; U.S. patent application Ser. No. 12/159,589, filed Aug. 15, 2008; and U.S. patent application Ser. No. 11/993,375, filed Feb. 6, 2009, the disclosures of which are each incorporated by reference herein in their entirety.

A method for surgically repairing a pelvic floor defect will now be described. A patient is catheterized and placed in the dorsal lithotomy position. Small incisions are made in the anterior or posterior vaginal wall (dependent on the repair compartment) through the vaginal mucosa and fascia and into the plane beyond. Advantageously, a thick dissection is created, leaving as much endopelvic fascia on the mucosa as possible. Tissue is dissected laterally and proximally to the sacrospinous ligament on both sides of the spine. The sacrospinous ligament is cleared of any excess fascia or muscle.

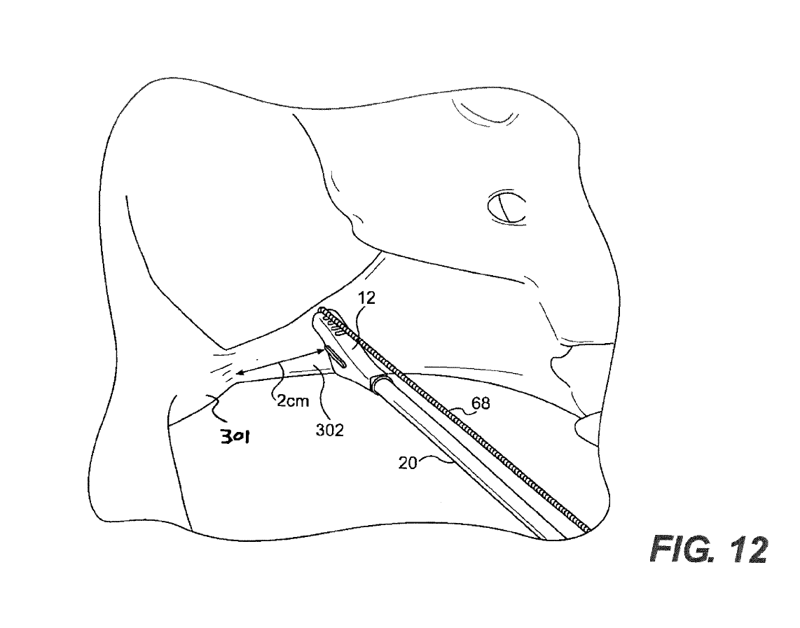

The distal end 70/102 of the first proximal implant arm 68/100 is attached to the needle housing 12 as shown in FIGS. 2F and 2G. The needle housing 12 is inserted into the vaginal dissection and positioned at the level of the sacrospinous ligament 302, approximately 2 cm medial to the ischial spine 301 (FIG. 12). Direct pressure is applied to the tactile feature 56 (FIG. 2D) on the needle housing 12. Trigger 29 (FIG. 1) is fully depressed, and then released. This action drives the bullet needle 72 and trailing implant arm 70/102 through the sacrospinous ligament, and into the needle catch 38. The needle housing 12 is withdrawn from the vaginal incision. Needle release tab 39 is depressed, releasing the bullet needle from the needle catch (FIG. 2H).

Care should be taken to ensure the tubular mesh arm remains on the underside of the support member 86/116. A hemostat can be clipped close to the end of the implant arm and clipped to the drape close to the vaginal incision. The entire process is then repeated on the contralateral side.

Once both proximal arms 68/100 have been drawn through the sacrospinous ligament 302, traction is applied to draw the support member 86/116 into place. The midline indicator 84/118 is used to ensure the support member 86/116 is positioned appropriately.

If the anterior implant 60 is being implanted, then the process for introducing distal arms 74 is as follows. A 1 cm vertical incision is made approximately 1 cm below the superior medial border of the obturator fossa and lateral to the bladder neck for the distal arm 74 of the implant 60. Ensure that the loop 176 is fully withdrawn into shaft 184 of introducer 168 by sliding button 172 to its distal-most location. Tip 180 of introducer 168 is inserted into the groin incision and gently punctures through the obturator membrane. The introducer 168 is oriented so that shaft 184 is on a horizontal plane, and the tip 180 is directed towards the level of the bladder neck. A finger is inserted into the vaginal incision, and is used to guide introducer tip 180 through the obturator internus, exposing at least 1-2 cm of the tip. A cytoscopy should be performed to confirm integrity of the bladder after the implant 60 has been positioned.

If the posterior implant 96 is being implanted, then the process for introducing distal arms 108 is as follows. A 1 cm vertical pararectal incision is made approximately 3 cm lateral and 3 cm inferior to the anus. Ensure that the loop 176 is fully withdrawn into shaft 184 of introducer 168 by sliding button 172 to its distal-most location. The tip 180 of introducer 168 is inserted into the pararectal incision, and oriented towards the vaginal introitus. Care should be taken to stay lateral to the anal sphincter and rectum during passage. A finger is inserted into the vaginal incision, and is used to guide the introducer tip 180 through the posterior vaginal wall incision at the perineal body at the most lateral portion of the dissection (the junction of the transverse perineal and bulbocavernosus muscles), exposing at least 1-2 cm of the introducer tip 180. A digital rectal examination should be performed to confirm integrity of the rectum after the implant 96 is positioned.

Whether the anterior implant 60 or posterior implant 96 is implanted, the following procedure for placing the distal arms is as follows. The introducer loop 176 is fully exteriorized from the vaginal introitus by sliding button 172 proximally. The distal tip 76/110 of the distal arm 74/108 is passed into loop 176 up to the fold 78/112 (about 4 cm). The loop 176 is retracted by moving button 172 to its proximal-most position. Introducer 168 is then retracted to draw the mesh arm out through the skin incision. Button 172 is moved to its distal-most position, and the distal arm 74/108 is removed from loop 176. The process is then completed on the contralateral side.

Next, traction is applied to distal arms 74/108 to draw implant 60/96 into position. The implant can be adjusted and/or trimmed such that the distal end of the midline indicator 84/118 is positioned next to the bladder neck (anteriorly), or perineal body (posteriorly).

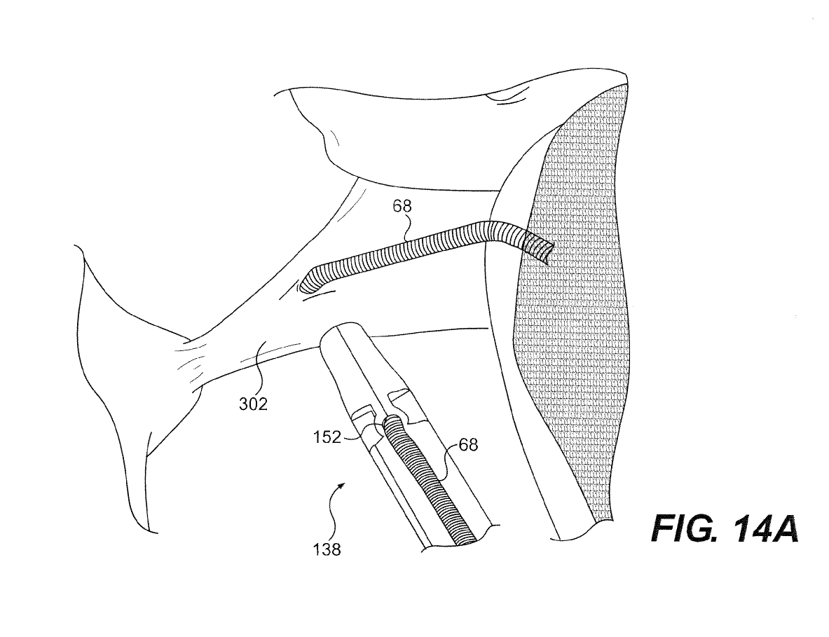

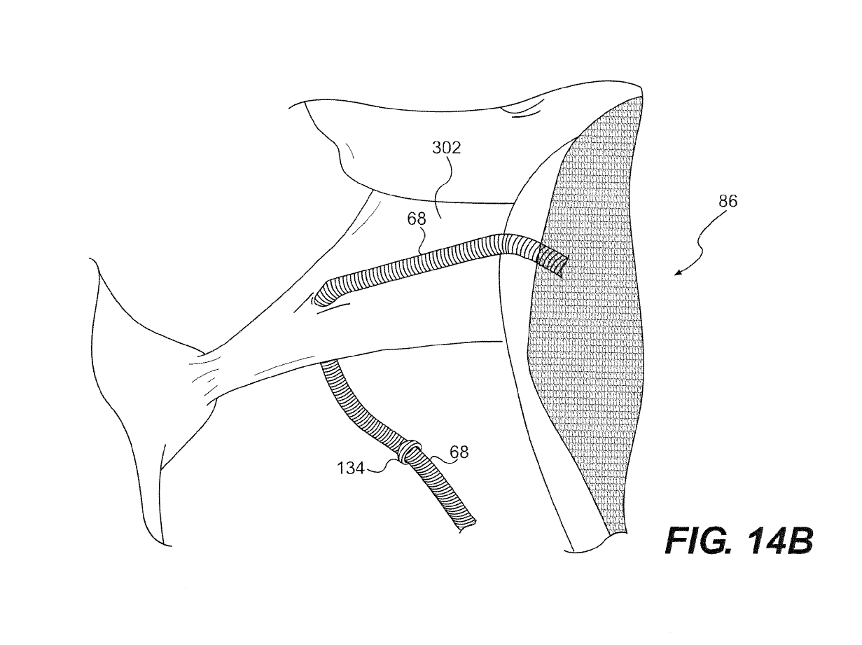

Following placement of the distal arms, the proximal arms can be placed as follows. Distal end 70/102 of an implant arm is threaded through deployment tool 138 as shown in FIG. 10. The deployment tool is slid down the implant arm until it reaches sacrospinous ligament 302 as shown in FIG. 14A. Button 140 is pressed to release securement ring 134 and trim excess mesh arm 68/100. The deployment device and the excess mesh are removed from the vaginal incision, and the process is repeated on the contralateral side.

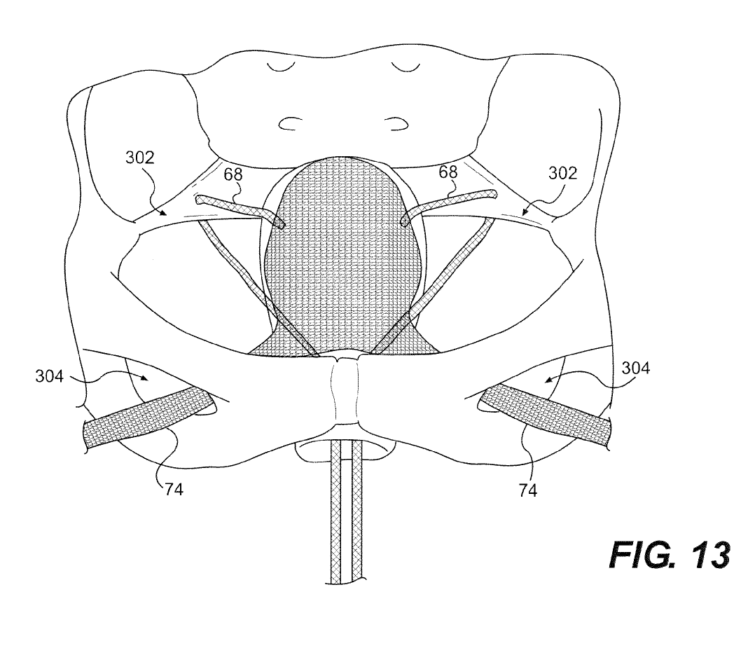

A plan view of an implanted anterior implant 60 is illustrated in FIG. 13. The proximal arms 68 have been drawn through the sacrospinous ligament 302 and out the vaginal incision, and the distal arms 74 have been drawn through the obturator foramen 304. Sutures can be used to secure the implant 60 to surrounding tissue.

According to various embodiments, the tension on the distal arms can be adjusted postoperatively. The mesh arms can be gently pushed to release a portion of the tension. The securement ring 134 on the proximal arms 68/100 will allow minor loosening of the arms while maintaining sufficient hold on the implant to ensure fixation.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1. A pelvic floor repair system comprising:
a) an introducer having a proximal end and a distal end, wherein a needle housing is located at the proximal end, a handle is located at the distal end, and a shaft connects the needle housing to the handle, the needle housing comprising:
(i) a curved needle carrier having a proximal end and a distal end, wherein the curved needle pivots about the center of its axis of rotation,
(ii) a first aperture from which the distal end of the needle carrier exits upon actuation, and
(iii) a second aperture;
the handle comprising a manual actuation mechanism that drives movement of the distal end of the needle carrier to exit from the first aperture toward the second aperture; and
b) an implant comprising a support portion and at least one extension connected to the support portion, the at least one extension having a proximal end connected to the support portion and a distal end connected to a needle.

2. The system according to claim 1, wherein the at least one extension comprises a tubular mesh arm defining a lumen extending at least partially therethrough.

3. The system according to claim 1, wherein the needle is a bullet needle having a distal end configured to pierce tissue, a shoulder, and a proximal end that receives the distal end of the at least one extension.

4. The system according to claim 3, wherein the proximal end of the bullet needle is crimped around the distal end of the at least one extension.

5. The system according to claim 1, wherein the second aperture includes a needle catch configured to releasably capture the needle.

6. The system according to claim 5, wherein the needle housing further comprises a manual release mechanism to release the needle from the needle catch.

7. The system according to claim 1, wherein the handle has a proximal end, a distal end, and a gripping region between the proximal and distal ends, the proximal end of the handle is connected to the distal end of the shaft, the shaft and the proximal end of the handle share the same longitudinal axis, and the longitudinal axis of the shaft and the longitudinal axis of the gripping region intersect at an angle ranging from 200° to 80°.

8. The system according to claim 1, further comprising:
(c) at least one introducer needle having a proximal end and a distal end and defining an inner lumen, the introducer needle having an opening that provides access to the inner lumen; and
(d) a drive wire terminating in an implant coupling element, the drive wire positioned within the inner lumen of the introducer needle, wherein the implant coupling element is extendable from the introducer needle opening to an extended position in which the implant coupling element is positioned outside of the inner lumen, and retractable to a retracted position in which the implant coupling element is positioned within the inner lumen.

9. The system according to claim 8, wherein the implant coupling element is a loop that can receive at least the distal end of the at least one extension.

10. The system according to claim 1, wherein the implant comprises two proximal extensions and two distal extensions, wherein at least two of the four extensions are each comprised of a tubular mesh defining a lumen extending at least partially therethrough.

11. The system according to claim 1, wherein the implant comprises two proximal extensions, each comprised of a tubular mesh defining a lumen extending at least partially therethrough; and two distal extensions, each comprised of a flat mesh.

12. The system according to claim 1, wherein the needle housing comprises a tissue engagement portion.

13. The system according to claim 12, wherein the tissue engagement portion has a concave shape that facilitates in-flow of tissue.

14. The system according to claim 13, wherein the concave shape is defined by an arc.

15. The system according to claim 14, wherein a line tangent to each end of the arc defines a first axis, and the shaft of the introducer defines a second axis.

16. The system according to claim 15, wherein the first axis intersects the second axis at an angle ranging from 15° to 80°.

17. The system according to claim 1, wherein the distal end of the needle carrier receives the proximal end of the needle.

18. The system according to claim 1, wherein the shaft comprises a bracket configured to receive and releasably secure the at least one implant extensions.

19. A pelvic floor repair system comprising:
a) an introducer having a proximal end and a distal end, wherein a needle housing is located at the proximal end, a handle is located at the distal end, and a shaft connects the needle housing to the handle; the needle housing comprising a curved needle carrier having a proximal end and a distal end, wherein the curved needle pivots about the center of its axis of rotation, the needle housing further comprising a first aperture from which the distal end of the needle carrier exits upon actuation, and a second aperture, the handle comprising a manual actuation mechanism that drives movement of the distal end of the needle carrier to exit from the first aperture toward the second aperture;
b) an implant comprising a support portion and at least one extension connected to the support portion, wherein the at least one extension has a proximal end connected to the support portion and a distal end connected to a needle; and
c) a locking feature associated with the at least one extension, the locking feature substantially locking the at least one extension in position relative to tissue.

20. The system according to claim 19, wherein the needle housing comprises at least one external feature that provides tactile feedback to the user.

21. The system according to claim 19, wherein the locking feature is a securement ring, through which the at least one extension is received.

22. The system according to claim 21, wherein the internal diameter of the securement ring comprises at least one feature that substantially permits movement of the extension through the ring in one direction, and substantially inhibits movement of the extension through the ring in the opposite direction.
